(19) United States
(12) Patent Application Publication
(10) Pub. No.: US 2026/0108398 A1
(43) Pub. Date: Apr. 23, 2026

(54) CONNECTING SHEET AND GARMENT ADHERING ARTICLE

(71) Applicant: 3M INNOVATIVE PROPERTIES COMPANY, St. Paul, MN (US)

(72) Inventors: Nobuharu Arai, Sagamihara-shi (JP); Marina Fukushi, Sagamihara-shi (JP)

(21) Appl. No.: 19/117,005

(22) PCT Filed: Sep. 29, 2023
(86) PCT No.: PCT/IB2023/059818

§ 371 Date: Mar. 28, 2025

(30) Foreign Application Priority Data
Sep. 30, 2022 (JP) 2022-158575

(51) Int. Cl. A61F 13/56; A44B 18/00; A61L 15/24

(52) U.S. Cl. CPC A61F 13/5611; A44B 18/0019; A61L 15/24

(57) ABSTRACT

To provide a connecting sheet with appropriate softness as well as a garment adhering article that can be attached to clothing. A connecting sheet connects with a target object by being superimposed on the target object, which includes: a sheet base material having a first main surface and a second main surface; a plurality of hook members protruding from the first main surface of the sheet base material in a first direction arranged mutually separated in a second direction in which the first main surface extends; and an adhesive layer adhered to the hook members, wherein the polyethylene content of the sheet base material and hook member is 15 weight percent or more, and the density of the polymer or polymer mixture of the sheet base material and the hook member is 0.925 g/cm³ or less.

### CROSS-REFERENCE TO RELATED APPLICATION

This application claims priority to JP Application No. 2022-158575, filed Sep. 30, 2022, the disclosure of which is incorporated by reference in its entirety herein.

### TECHNICAL FIELD

One form of the present invention relates to a connecting sheet and a garment adhering article.

### BACKGROUND

Conventionally, to fix an article to a garment or the like, a connecting sheet using an adhesive material is used. An absorbent article such as a napkin is fixed to garments such as underwear via the adhesive material of the connecting sheet. For example, JP 2010-115563 describes a connecting sheet having a slip-prevention adhesive layer for releasably attaching an article to garments. The connecting sheet has an adhesive layer on a main surface of a sheet base material.

### SUMMARY

Here, if the connecting member is too hard, it may cause dermatalgia when it touches the user's skin. Therefore, there is demand for a connecting sheet having an appropriate softness that can reduce dermatalgia. With regards to this type of issue, one resolution proposal is to make the sheet base material thinner, but this method tends to affect productivity and quality stability and leads to issues such as wrinkles and tearing of the connecting sheet. In addition, there is also a resolution proposal of making the hook members finer, but this type of method affects the characteristics of the connecting sheet.

A connecting sheet of an aspect of the present invention that connects with a target object by being superimposed on the target object, includes: a sheet base material having a first main surface and a second main surface; a plurality of hook members protruding from the first main surface of the sheet base material in a first direction arranged mutually separated in a second direction in which the first main surface extends; and an adhesive layer adhered to the hook members, wherein the polyethylene content of the sheet base material and hook member is 15 wt % or more, and the density of the polymer or polymer mixture of the sheet base material and the hook member is 0.925 g/cm³ or less.

With the present invention, a connecting sheet with appropriate softness and a garment adhering article can be provided.

### BRIEF DESCRIPTION OF THE DRAWINGS

FIG. 1 is a view illustrating a napkin including a connecting sheet in accordance with an embodiment;

FIG. 2 is a cross sectional view illustrating a part of the connecting sheet in accordance with the embodiment;

FIG. 3A is a diagram depicting a state of attaching a napkin to underwear;

FIG. 3B is a diagram depicting a state of a napkin attached to underwear;

FIG. 4 is a table indicating conditions for samples 1 to 14 prepared for measurement and the measurement results;

FIG. 5 is a graph depicting the relationship between PE content of each sample and softness;

FIG. 6 is a table indicating the PE content and mixed polymer density for each sample;

FIG. 7 is a plotted graph of the relationship between PE content and mixed polymer density for each sample;

FIG. 8 is a table describing the measurement method for mixed polymer density;

FIG. 9 is a graph for describing the PP-PE ratio measurement; and

FIG. 10 indicates the relationship of the ratio between PE content and peak strength.

### DETAILED DESCRIPTION

In the following, various embodiments will be described in detail with reference to drawings. Note that the same reference signs are used for the same or corresponding portions in the drawings.

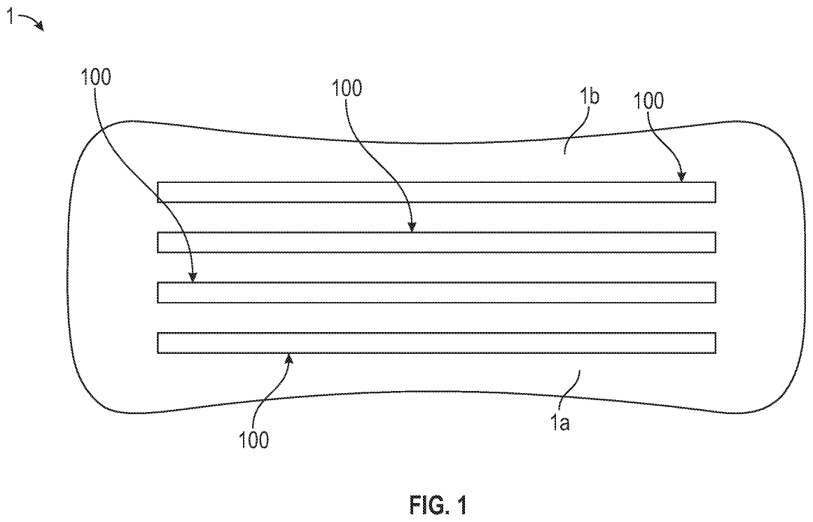

FIG. 1 is a view illustrating a napkin including a connecting sheet according to an embodiment of the present invention. A napkin 1 includes a front surface la that faces the skin and has liquid permeability, a back surface 1b (connecting surface) that faces a garment and has liquid impermeability, and connecting sheets 100 provided on the back surface 1b. The back surface 1b is a connecting surface connected to the garment. In the example illustrated in FIG. 1, the connecting sheets 100 are provided in the plurality of regions of the back surface 1b at intervals. Note that the number of regions where the connecting sheets 100 are provided, the shape of each region, and the dimension of each region are not limited thereto and can be designed as desired. In addition, the article to which the connecting sheets 100 are applied is not limited to the napkin, and may be applied to, for example, a light incontinence pad, a pocket warmer (stuck to garments), or the like. The napkins and light incontinence pads may be collectively referred to as absorbent articles. The absorbent articles are also referred to as garment adhering articles including a pocket warmer or the like stuck to garments.

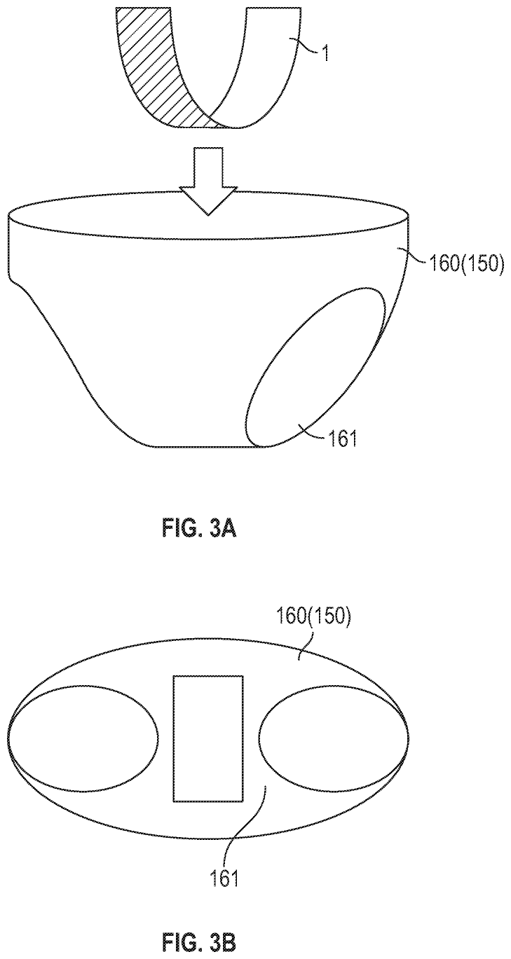

FIG. 3A is a top view depicting a state of the napkin 1 being attached to the target object 150. FIG. 3B is a diagram depicting a state of the napkin 1 attached to a target object 150. Panties 160 are an example of a target object 150. The napkin 1 is attached to the bottom part 161 of the panties 160.

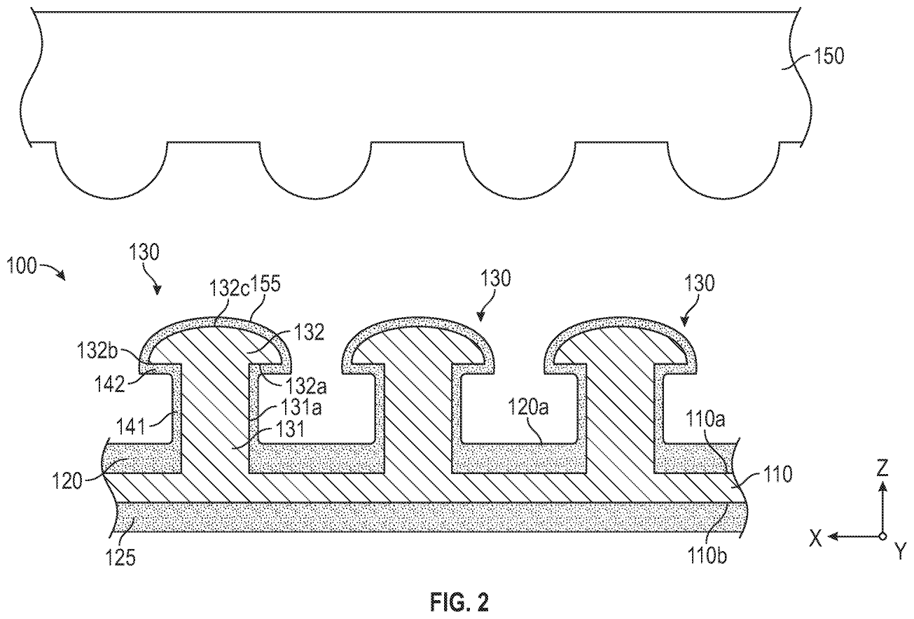

Next, a configuration of the connecting sheet 100 will be described with reference to FIG. 2. FIG. 2 is a cross sectional view illustrating a part of the connecting sheet 100 in accordance with an embodiment. For the purpose of explanation, FIG. 2 also illustrates the XYZ Cartesian coordinate system.

The connecting sheet 100 is stacked on a target object 150 such as underwear and connected to the target object 150. As a result, the connecting sheet 100 can fix the napkin 1 to the target object 150 such as a garment. As illustrated in FIG. 2, the connecting sheets 100 each include a sheet base material 110, a first adhesive layer 120, a second adhesive layer 125, and a plurality of hook members 130. In the present embodiment, a direction that is orthogonal to the sheet base material 110 is defined as a Z-axis direction (first direction). A direction that is orthogonal to the Z-axis direction is defined as an X-axis direction, and a direction that is orthogonal to the Z-axis direction and the X-axis direction is defined as a Y-axis direction.

The sheet base material 110 has a substantially sheet-like shape and has a first main surface 110a and a second main surface 110b that are opposed to each other in the Z-axis direction. The sheet base material 110 extends in an XY-plane direction (second direction). In other words, the first main surface 110a and the second main surface 110b extend in the XY-plane direction. The plurality of hook members 130 are formed on the first main surface 110a.

The first adhesive layer 120 is formed on the first main surface 110a of the sheet base material 110. The first adhesive layer 120 is provided so as to fill spaces between the plurality of hook members 130. The first adhesive layer 120 is formed so as to cover the substantially entire surface of the first main surface 110a (except for the positions of the hook members 130). Examples of the material for the first adhesive layer 120 include natural rubber/resin copolymers, synthetic rubber/resin copolymers, and acrylate copolymers. The thickness of the first adhesive layer 120 may be 10 micrometers (um) or more, or 20 μm or more, and may be 100 μm or less, or 90 μm or less. The applied amount may be 10 grams per square meter (gsm) or more, or 20 gsm or more, and may be 100 gsm or less, or 90 gsm or less. In the state of an adhesive state, the adhesive strength of the first adhesive layer 120 may be 2 Newtons per 25 millimeters (N/25 mm) or more or 4 (N/25 mm) or more and may be 14 (N/25 mm) or less or 8 (N/25 mm) or less. The adhesive strength of the first adhesive layer 120 is a value defined by the method JISZ 0237.

The second adhesive layer 125 is formed on the second main surface 110b of the sheet base material 110. The material for the second adhesive layer 125 is the same adhesive material as the material for the first adhesive layer 120. The first adhesive layer 120 and the second adhesive layer 125 may be formed from the same adhesive material or may be formed from different adhesive materials. The second adhesive layer 125 may be omitted. The thickness of the first adhesive layer 120 may be 10 μm or more or 20 μm or more and may be 120 um or less or 110 um or less.

The hook members 130 protrude from the first main surface 110a of the sheet base material 110 in the Z-axis direction (first direction). The hook members 130 are spaced at intervals in the XY-plane direction in which the first main surface 110a extends. The hook members may be arranged in a grid pattern that is perpendicular to the XY-plane direction and may be arranged in a grid pattern that is oblique from the XY-plane direction, a staggered pattern, a random pattern, and the like. The hook members 130 protrude from the front surface 120a on the Z-axis positive side of the first adhesive layer 120 in the Z-axis direction.

The hook members 130 each include a column part 131 and a head part 132. The column part 131 is a portion that extends from the sheet base material 110 in the Z-axis direction and protrudes from the front surface 120a of the first adhesive layer 120. The head part 132 is a part that spreads from a tip of the column part 131 in the XY-plane direction. Note that the shape of the column part 131 and the head part 132 of all of the hook members 130 in the connecting sheet 100 may be the same (variations in the range of manufacturing errors are allowable) or may be different in different regions.

The hook member 130 includes adhesive material applied parts 141, 142, 155 formed by covering a portion protruding from the first adhesive layer 120 with an adhesive material. Specifically, an outer circumferential surface 131a of the column part 131 is covered with the adhesive material. As a result, the adhesive material applied portion 141 is formed on the outer circumference of the column part 131. Further, a lower surface 132a of the head part 132 is covered with the adhesive material. As a result, the adhesive material applied portion 142 is formed on the lower side of the head part 132. The adhesive material applied portion 142 is formed across the entire region between an inner circumferential end (a connection portion of the column part 131 and the head part 132) and an outer circumferential end 132b on the lower surface 132a of the head part 132. Accordingly, the adhesive material applied portion 142 of the head part 132 further extends to the outer circumferential side than the adhesive material applied portion 141 of the column part 131. An upper surface 132c of the head part 132 is covered with an adhesive. As a result, the adhesive material applied part 155 is formed on the upper side of the head part 132 Next, materials for the sheet base material 110 and the hook member 130 will be described. The sheet base material 110 and the hook member 130 contain at least polyethylene (PE). The sheet base material 110 and the hook member 130 are composed of, for example, a mixed polymer obtained by adding an additive polymer to a base polymer. Polypropylene (PP) or polypropylene-polyethylene (PP-PE) copolymer can be utilized as the base polymer. Low density polyethylene can be utilized as the additive polymer. The base polymer of the mixed polymer tends to be stiff due to high crystallization of the polymer. On the other hand, low-density resins tend to have low rigidity (soft) due to low crystallization of the polymer. Adjusting the amount of low-density polyethylene added to the base polymer adjusts the polyethylene content and density in the mixed polymer. Polyethylene monomers are preferably derived from plants (sugar cane, and the like).

The polyethylene content of the sheet base material 110 and the hook member 130 is 15 weight percent (wt %) or more and 100 wt % or less, and the density of the mixed polymer of the sheet base material 110 and the hook member 130 is 0.900 grams per cubic centimeter (g/cm³) or more and 0.925 g/cm³ or less. In addition, the polyethylene content of the sheet base material 110 and the hook member 130 may be 20 wt % or more and 95 wt % or less, and the density of the mixed polymer of the sheet base material 110 and the hook member 130 may be 0.901 g/cm³ or more and 0.920 g/cm³ or less. In addition, the polyethylene content of the sheet base material 110 and the hook member 130 may be 25 wt % or more and 60 wt % or less, and the density of the mixed polymer of the sheet base material 110 and the hook member 130 may be 0.902 g/cm³ or more and 0.915 g/cm³ or less.

The thickness of the sheet base material 110 may be 70 μm or less or 50 μm or less. In addition, the thickness of the sheet base material 110 may be 10 μm or more or 20 μm or more. The average height of the hook member 130 from the first main surface 110a may be 170 μm or more or 90 μm or more. The average height of the hook member 130 from the first main surface 110a may be 330 μm or less or 310 μm or less. The diameter of the column part 131 of the hook member 130 may be 100 μm or more and may be 150 μm or more. The diameter of the column part 131 of the hook member 130 may be 350 μm or less and may be 250 μm or less. In addition, the density in which the hook members 130 are arranged may be, for example, approximately 500 to 5000 per square inch (77.5 to 775 per square centimeter) or approximately 1600 to 3500 per square inch (248 to 542 per square centimeter).

The shear force of the connecting sheet 100 connected to the target object 150 is a parameter representing the difficulty of displacement of the napkin 1 connected to the object 150 via the connecting sheet 100. The shear force of the connecting sheet 100 connected to the target object 150 may be 0.9 N/5 cm² or more and may be 2.4 N/5 cm² or more. In addition, the shear force of the connecting sheet 100 connected to the target object 150 may be 9 N/⁵ cm² or less and may be 8 N/5 cm² or less.

The initial tack of the connecting sheet 100 is a parameter indicating sticky feel. The initial tack may be 0.01 N/6.25 cm² or more and may be 0.02 N/6.25 cm² or more. The initial tack may be 0.1 N/6.25 cm² or less and may be 0.09 N/6.25 cm² or less.

As a measurement method for measuring peel force for each sample from underwear, underwear was secured to a stainless steel panel and then a sample was placed and then roller pressure of 7 kilograms (kg) was reciprocated once and measurement was performed by peeling performed at a peel speed of 300 millimeters (mm)/minute and 90°. The surface of underwear “PGG340” manufactured by WACOAL HOLDINGS CORP. that was in contact with skin was used as the target object for this measurement. The size of the sheet base material was 25 mm×20 mm.

In addition, as dynamic shear strength, underwear was secured to a stainless steel panel, shear was caused by pulling at a 180° direction (direction along 25 mm side) with a crosshead speed of 30 mm/minute after one reciprocation of a 2 kg roller pressure, and the tensile strength was measured at that time. The surface of underwear “EC4033” manufactured by WACOAL HOLDINGS CORP. that was in contact with skin was used as the target object for this measurement. The size of the sheet base material was 25 mm×20 mm.

In addition, a stainless steel T-shaped block was provided for the initial tack measurement. The pressing surface of the T-shaped block was set to a size of 25 mm×25 mm. The pressing surface of the T-shaped block is pressed onto the hook member 130 covered with adhesive. Herein, with the measurement value of a force when a T-shaped block is pressed upward at 300 mm/min by pressing 2 kg ×5 seconds on a hook member 130 without adhesive as a reference value, the difference in a measurement value of a force when a T-shaped block is pressed upward at 300 mm/min by pressing 2 kg ×5 sec on the hook member 130 when covered with adhesive and this reference value was calculated as the initial tack.

The connecting sheet 100 may have a softness of 0.06 gram-force(gf)*cm²/cm or less and may be 0.05 gf*cm²/cm or less. The softness of the connecting sheet 100 may be 0.01 gf*cm²/cm or more and may be 0.02 gf*cm²/cm or more. The softness of the connecting sheet 100 is measured by a series of devices called KES (Kawabata Evaluation System). The results measured by KES indicate the bendability of the connecting sheet 100.

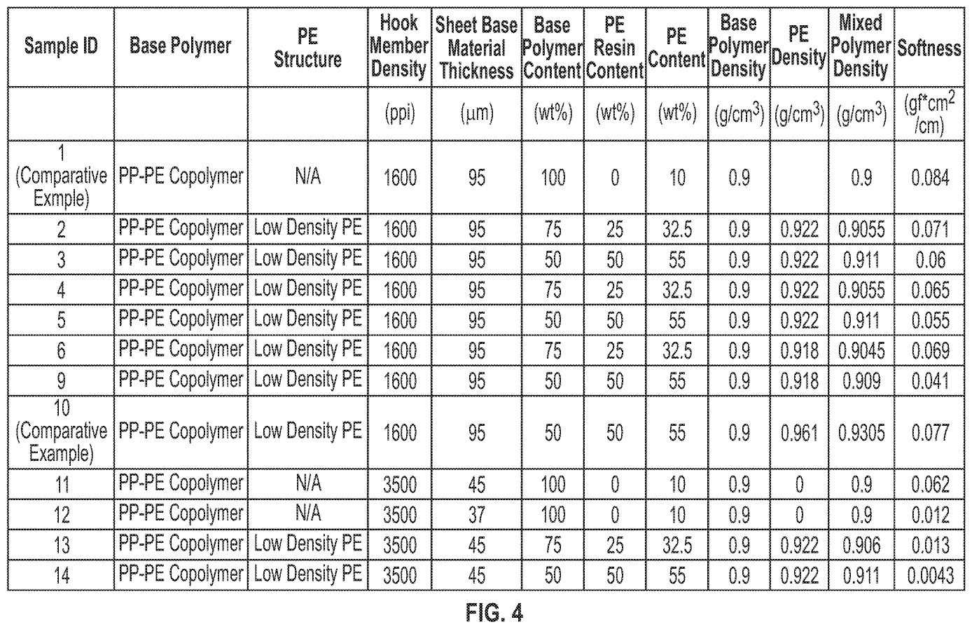

Next, with reference to FIG. 4 to FIG. 10, the relationship between the softness of the connecting sheet 100 and the content and density of polyethylene in the polymer mixture will be described. FIG. 4 is a table depicting conditions of samples 1 to 14 prepared for measurement and the measurement results. The height of the hook member was 420 μm, and the diameter of the head part was 290 μm. “Quintac 3620/Quintone M100//Irganox1076=60/39/1” was used as the adhesive. The amount of adhesive applied was set to 93 g/m².

In the table of FIG. 4, the base polymer used for each sample is noted in the “base polymer” column. The additional polymer added is noted in the “PE structure” column. “Hook member density” notes the density of the hook member. The thickness of the sheet base material is noted in the “sheet base material thickness” column. With the overall polymer mixture as 100 wt %, the base polymer content is noted in the “base polymer content” column. With the overall polymer mixture as 100 wt %, the added polymer PE content is noted in the “PE resin content” column. With the overall polymer mixture as 100 wt %, the PE content is noted in the “PE content” column. The “base polymer density” column notes the density of the base polymer. The “PE density” column notes the density of the added polymer PE. The “mixed polymer density” column notes the density of the mixed polymer as a whole. The “softness” column notes the measurement results for softness.

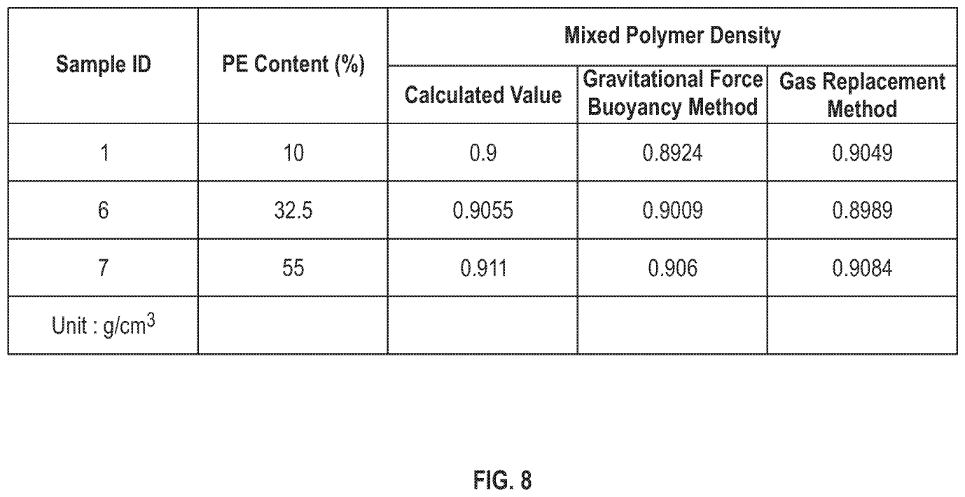

Of the items in the table described above, the density measurement method will be described with reference to FIG. 8. FIG. 8 indicates a measurement method of a mixed polymer density with regards to samples 1, 6 and 7. The mixed polymer density utilizes a calculated value of the average measurement value using the gravitational force buoyancy method and the measurement value using the gas replacement method. The gravitational force buoyancy method is a method for measuring the density based on the Archimedes principle of an object in a liquid receiving a buoyancy force equivalent to the weight of the liquid displaced by the object. Air integrated in the sample is a cause of variation for this method, leading to error within 1%. The gas replacement method is a measurement method of the density that uses Boyle's law (PV=constant).

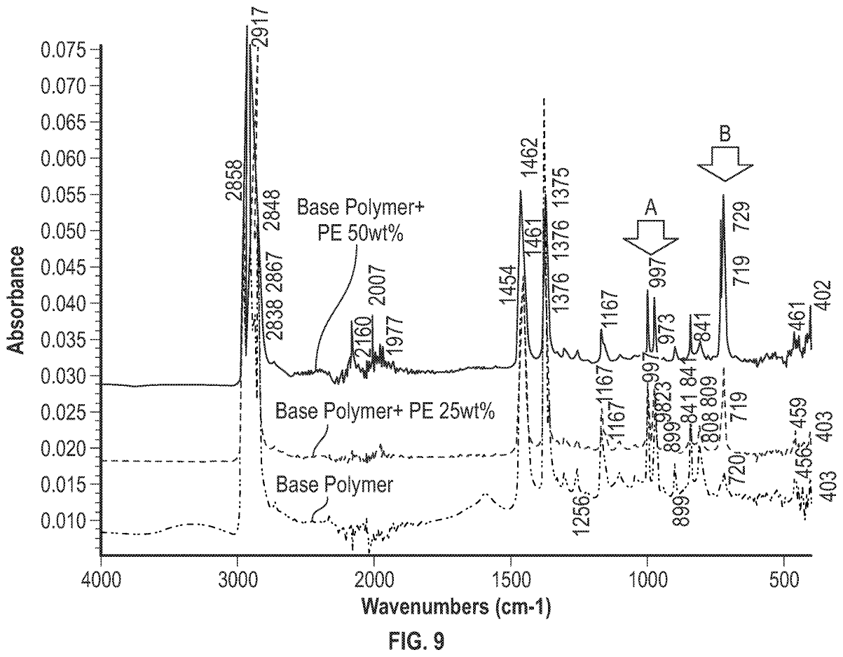

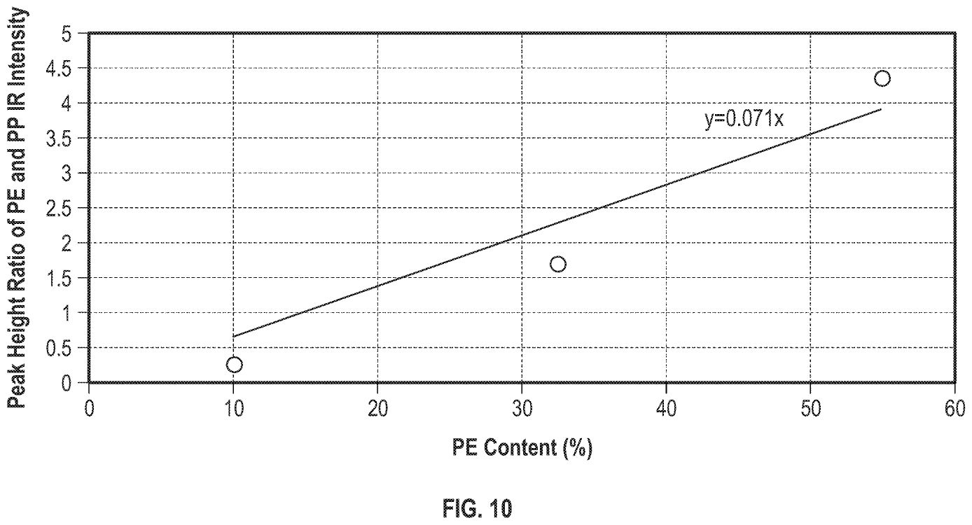

Of the items in the table described above, the measurement method of the ratio of PP and PE will be described with reference to FIG. 9 and FIG. 10. In FIG. 9, infrared spectroscopy (IR) measurements were made for a “base polymer+added polymer PE at 50 wt %” sample, a “base polymer+added polymer PE at 25 wt %” sample, and a “base polymer”. As a result, a PP derived peak positioned at 997 cm⁻¹ was observed (“A” in figure) and a PE derived peak positioned at 720 cm⁻¹ was observed (“B” in figure). The intensity of these PP derived peaks and the intensity of the PE derived peaks were obtained and the ratio of the two was calculated. FIG. 10 depicts the relationship between the PE content and peak intensity ratio.

A method for measuring softness will be described. Each sample was made into a 25 mm×25 mm test piece, set in a bending device (Kawabata Evaluation System (KES)), and the softness of each sample was measured.

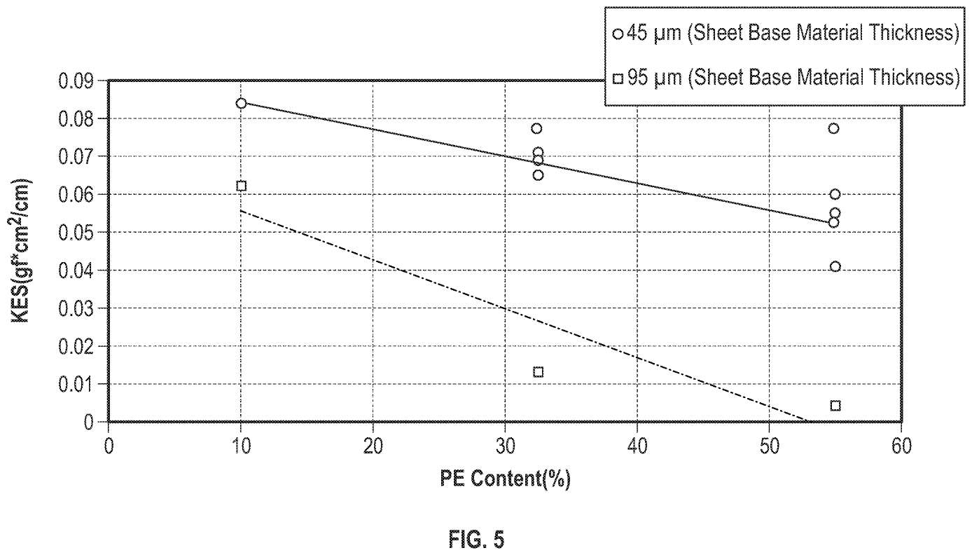

FIG. 5 depicts the relationship between the PE content of the added polymer and the softness measurement results. FIG. 5 compares the results when the sheet base material thickness was 95 μm and the results when the sheet base material thickness was 45 μm. It was confirmed that increasing PE content improved softness of the connecting sheet regardless of the sheet base material thickness.

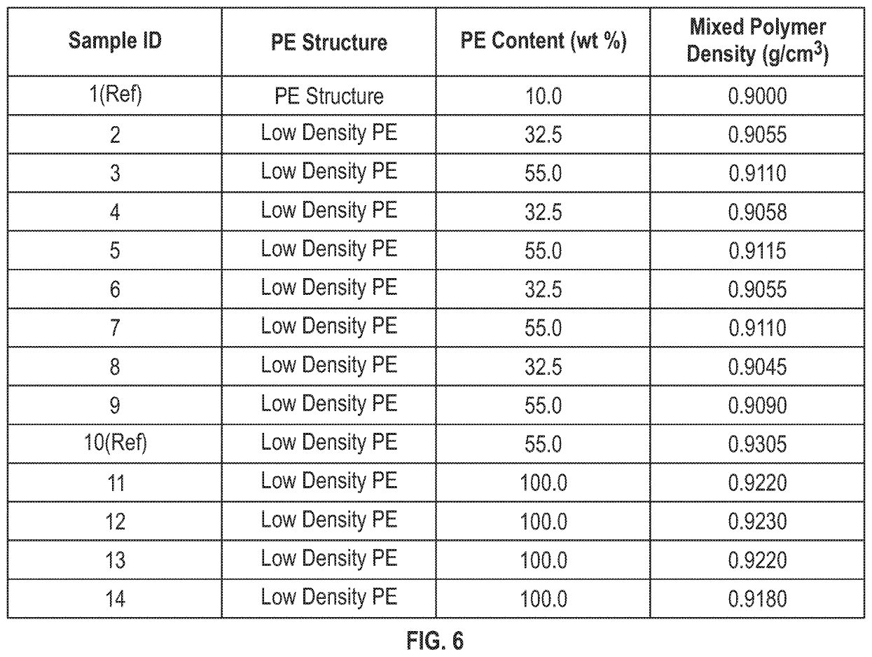

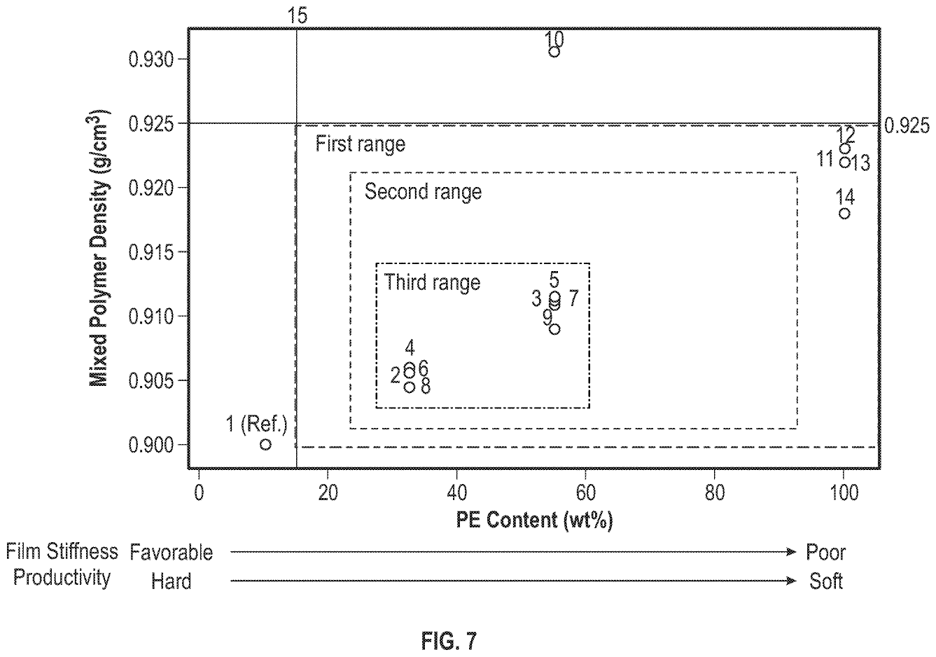

FIG. 6 indicates the PE structure for each sample, PE content, and mixed polymer density. FIG. 7 is a plotted graph of the relationship between PE content and mixed polymer density for each sample. PE content and mixed polymer density preferred range are set based on the plot results of each sample. FIG. 7 depicts a first range indicating a preferred range, a second range indicating a more preferred range, and a third range indicating an even more preferred range. The PE content for the first range is 15 wt % or more and 100 wt % or less. The mixed polymer density for the first range is 0.900 g/cm³ or more and 0.925 g/cm³ or less. The PE content for the second range is 20 wt % or more and 95 wt % or less. The mixed polymer density for the second range is 0.901 g/cm³ or more and 0.920 g/cm³ or less. In addition, the PE content for the third range is 25 wt % or more and 60 wt % or less. The mixed polymer density for the third range is 0.902 g/cm³ or more and 0.915 g/cm³ or less.

Next actions and effects of the connecting sheet 100 in accordance with the present embodiment will be described.

A connecting sheet 100 that connects with a target object 150 by being superimposed on the target object 150, includes: a sheet base material 110 having a first main surface 110a and a second main surface 110b; a plurality of hook members 130 protruding from the first main surface 110a of the sheet base material 110 in a first direction arranged mutually separated in a second direction in which the first main surface 110a extends; and an adhesive layer 120 adhered to the hook members 130, wherein the polyethylene content of the sheet base material 110 and hook member 130 is 15 wt % or more, and the density of the polymer mixture of the sheet base material 110 and the hook member 130 is 0.925 g/cm³ or less.

The connecting sheet 100 includes a plurality of hook members 130 protruding from the first main surface 110a of the sheet base material 110 in the first direction and the adhesive layer 120 adhered to the hook members 130. Therefore, the connecting sheet 100 is connected to the target object 150 by the adhesiveness of the hook members 130 and the adhesive layer 120. Here, the polyethylene content of the sheet base material and the hook members is 15 wt % or more and the mixed polymer density of the sheet base material and the hook member is 0.925 g/cm³ or less. In this case, adjusting the polyethylene content and the mixed polymer density enables achieving an appropriate softness of the sheet base material and the hook members. Thus, a connecting sheet with suitable softness can be provided.

In some embodiments, the polyethylene content of the sheet base material 110 and the hook members 130 is 20 to 95 wt %, and the mixed polymer density of the sheet base material 110 and the hook members 130 is 0.901 to 0.920 g/cm³.

The thickness of the sheet base material may be 70 μm or less. In this case, it is possible to suppress hardening of the connecting sheet due to excessive thickness of the sheet base material.

The shear force of the connecting sheet connected to the target object may be 0.9 N/⁵ cm2 or more. In this case, the connecting sheet can be used to prevent the garment adhering article attached to clothing connected to the target object from shifting.

Initial tack may be between 0.01 and 0.1 N/6.25 cm². In this case, the connecting sheet can be provided with appropriate feel.

The softness may be 0.06 gf*cm²/cm or less. In this case, the softness of the connecting sheet can be set within an appropriate range.

The garment adhering article is provided with the aforementioned connecting sheet on the connecting surface connected to the garment. In this case, when the connecting sheet touches the user's skin, damage to the skin can be reduced.

The present invention is not intended to be limited to the embodiments described above.

For example, the shape of the hook members is not limited to the embodiment described above and may be any shape. In other words, as long as the hook member has the column part and the head part and the adhesive material layer can be formed on the lower surface of the head part, the shape of the hook member is not particularly limited.

### Aspect 1

A connecting sheet that connects with a target object by being superimposed on the target object, includes:
a sheet base material having a first main surface and a second main surface;
a plurality of hook members protruding from the first main surface of the sheet base material in a first direction arranged mutually separated in a second direction in which the first main surface extends; and
an adhesive layer adhered to the hook members; wherein the polyethylene content of the sheet base material and hook member is 15 wt % or more, and
the density of the polymer mixture of the sheet base material and the hook member is 0.925 g/cm³ or less.

### Aspect 2

The connecting sheet according to aspect 1, wherein the polyethylene content of the sheet base material and the hook member is 20 to 95 wt % and the density of the polymer mixture of the sheet base material and the hook member is 0.901 to 0.920 g/cm³.

### Aspect 3

The connecting sheet according to aspect 1 or 2, wherein the thickness of the sheet base material is 70 μm or less.

### Aspect 4

The connecting sheet according to any one of aspects 1 to 3, wherein the shear force of the connecting sheet connected to the target object is 0.9 N/5 cm² or more.

### Aspect 5

The connecting sheet according to any one of aspects 1 to 4, having an initial tack of 0.01 to 0.1 N/6.25 cm².

### Aspect 6

The connecting sheet according to any one of aspects 1 to 5, having a softness of 0.06 gf*cm²/cm or less.

### Aspect 7

A garment adhering article comprising the connecting surface described in any one of aspects 1 to 6 on a surface connected to a garment.

### REFERENCE SIGNS LIST

1. Napkin, 100. Connecting sheet, 110. Sheet base material, 110a. First main surface, 110b. Second main surface, 120. First adhesive layer, 125. Second adhesive layer, 130. Hook member, 150. Target object

1. A connecting sheet that connects with a target object by being superimposed on the target object, comprising:
a sheet base material having a first main surface and a second main surface;
a plurality of hook members protruding from the first main surface of the sheet base material in a first direction arranged mutually separated in a second direction in which the first main surface extends; and
an adhesive layer adhered to the hook members, wherein polyethylene the content of the sheet base material and hook member is 15 weight percent or more, and the density of the polymer or polymer mixture of the sheet base material and the hook member is 0.925 g/cm³ or less.

2. The connecting sheet of claim 1, wherein the density of the polymer or polymer mixture of the sheet base material and the hook member is 0.900 g/cm³ or more.

3. The connecting sheet of claim 1, wherein the polymer mixture comprises polyethylene and polypropylene.

4. The connecting sheet of claim 1, wherein the polyethylene content of the sheet base material and the hook member is in a range from 20 weight percent to 95 weight percent.

5. The connecting sheet of claim 1; wherein the density of the polymer or polymer mixture of the sheet base material and the hook member is in a range from 0.901 g/cm³ to 0.920 g/cm³.

6. The connecting sheet of claim 1, wherein the polyethylene content of the sheet base material and the hook member is 25 weight percent or more and 60 weight percent or less.

7. The connecting sheet of claim 1, wherein the density of the polymer or polymer mixture of the sheet base material and the hook member is 0.902 g/cm³ or more and 0.915 g/cm³ or less.

8. The connecting sheet of claim 1, wherein the thickness of the sheet base material is 70 μm or less.

9. The connecting sheet of claim 1, wherein the shear force of the connecting sheet connected to the target object is 0.9 N/⁵ cm2 or more.

10. The connecting sheet of claim 1, having an initial tack of 0.01 N/6.25 cm² to 0.1 N/6.25 cm².

11. The connecting sheet of claim 1, having a softness of 0.06 gf*cm²/cm or less.

12. A garment-adhering article comprising the connecting surface of claim 1 on a surface connected to a garment.

13. The garment-adhering article of claim 12, wherein the garment-adhering article is an absorbent article or a pocket warmer.

14. The garment-adhering article of claim 12, wherein the garment-adhering article is a napkin or an incontinence pad.

15. The connecting sheet of claim 11, wherein the polyethylene content of the sheet base material and the hook member is 25 weight percent or more and 60 weight percent or less.

16. The connecting sheet of claim 11, wherein the polymer mixture comprises polyethylene and polypropylene.

17. The connecting sheet of claim 11, wherein the thickness of the sheet base material is 70 μm or less.

18. The connecting sheet of claim 8, wherein the polymer mixture comprises polyethylene and polypropylene.

19. The connecting sheet of claim 8, wherein the polyethylene content of the sheet base material and the hook member is in a range from 20 weight percent to 95 weight percent.

20. The connecting sheet of claim 8, wherein the polyethylene content of the sheet base material and the hook member is 25 weight percent or more and 60 weight percent or less.
